(19) United States
(12) Patent Application Publication
(10) Pub. No.: US 2021/0290901 A1
(43) Pub. Date: Sep. 23, 2021

(54) TUBULAR INSTRUMENT HAVING A DYNAMIC TIP AND RELATED DEVICES AND METHODS

(71) Applicant: Becton, Dickinson and Company, Franklin Lakes, NJ (US)

(72) Inventors: Jonathan Karl Burkholz, Salt Lake City, UT (US); S. Ray Isaacson, Layton, UT (US); Joseph Spataro, Cottonwood Heights, UT (US); Weston F. Harding, Lehi, UT (US); Yiping Ma, Layton, UT (US)

(21) Appl. No.: 17/195,319

(22) Filed: Mar. 8, 2021

Related U.S. Application Data
(60) Provisional application No. 62/993,456, filed on Mar. 23, 2020.

(51) Int. Cl. A61M 25/00

(52) U.S. Cl. CPC A61M 25/0075; A61M 2205/02; A61M 25/007

(57) ABSTRACT

A delivery device to deliver a tubular instrument into a catheter extending distally from a catheter adapter. The delivery device may include a housing configured to couple to the catheter adapter. The delivery device may include a tubular instrument configured to insert through the catheter. The tubular instrument may include a distal end, a proximal end, and a lumen extending between the distal end and the proximal end. The distal end may include a distal tip, which may be closed. The proximal end may be secured within the housing. The tubular instrument may be configured to advance distally with respect to the housing. The delivery device may include a pressure-sensitive valve disposed at the distal tip or within the distal end. The pressure-sensitive valve may be configured to move from the closed position to an open position in response to a predetermined vacuum pressure level within the lumen.

### RELATED APPLICATIONS

This application claims the benefit of U.S. Provisional Patent Application No. 62/993,456, filed on Mar. 23, 2020, entitled “TUBULAR INSTRUMENT HAVING A DYNAMIC TIP AND RELATED DEVICES AND METHODS,” which is incorporated herein in its entirety.

### BACKGROUND

A catheter is commonly used to infuse fluids into vasculature of a patient. For example, the catheter may be used for infusing normal saline solution, various medicaments, or total parenteral nutrition. The catheter may also be used for withdrawing blood from the patient.

The catheter may include an over-the-needle peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Blood withdrawal using the catheter may be difficult for several reasons, particularly when a dwell time of the catheter within the vasculature is more than one day. When the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, the catheter is often used for acquiring a blood sample at a time of catheter placement, but the catheter is less frequently used for acquiring a blood sample during the catheter dwell period. Therefore, when a blood sample is required, an additional needle stick is often needed to provide vein access for blood collection, which may be painful for the patient and result in higher material costs.

In some instances, in order to avoid the additional needle stick, a tubular instrument may be used to access the vasculature of the patient via the catheter. The tubular instrument may be inserted through the catheter and into the vasculature to extend a life of the catheter and allow blood withdrawal through the catheter without the additional needle stick.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices. In particular, the present disclosure relates to a tubular instrument and related devices and methods. In some embodiments, a delivery device to deliver a tubular instrument into a catheter may facilitate an increased dwell period of the catheter. In further detail, in some embodiments, the delivery device may be used to advance the tubular instrument into the catheter and/or beyond a distal tip of the catheter for fluid infusion or blood draw when the catheter is compromised or nearing an end of its life. In some embodiments, the tubular instrument may be configured to reduce trauma to a vein of a patient upon contact with the vein of the patient, compared to prior art devices. In some embodiments, the tubular instrument may provide gentle, soft contact between the tubular instrument and a vein wall, which may reduce trauma to the vein wall. In some embodiments, the tubular instrument may include another catheter. In some embodiments, the tubular instrument may include a probe.

In some embodiments, the delivery device may include a housing configured to couple to a catheter adapter. In some embodiments, the catheter may extend distally from the catheter adapter. In some embodiments, the delivery device may include the tubular instrument, which may be configured to insert through the catheter. In some embodiments, the tubular instrument may include a distal end, a proximal end, and a lumen extending between the distal end and the proximal end. In some embodiments, the distal end of the tubular instrument may include a distal tip, which may be closed. In some embodiments, the proximal end of the tubular instrument may be secured within the housing. In some embodiments, the tubular instrument may be disposed within the housing. In some embodiments, the tubular instrument may be configured to advance distally with respect to the housing.

In some embodiments, the delivery device may include a dynamic valve, which may open and/or close in response to a user input. For example, the delivery device may include a pressure-sensitive valve, which may be in a closed position. In some embodiments, the pressure-sensitive valve may be configured to move from the closed position to an open position in response to a predetermined pressure differential within the lumen of the tubular instrument. In some embodiments, the pressure-sensitive valve may be in the closed position to prevent fluid from flowing through the lumen. In some embodiments, the pressure-sensitive valve may be in the closed position under normal physiological pressures.

In some embodiments, the pressure-sensitive valve may be disposed at the distal tip. In these and other embodiments, the pressure-sensitive valve may include a slit. In some embodiments, the pressure-sensitive valve may include a cross slit. In some embodiments, a length of the cross slit may extend across an entirety of a width of the distal end. In some embodiments, a length of the cross slit may extend only partially across a width of the distal end of the tubular instrument. In some embodiments, the tubular instrument may include multiple holes within the distal end and proximal to the pressure-sensitive valve. In some embodiments, the pressure-sensitive valve may be monolithically formed with the tubular instrument as a single unit.

In some embodiments, the pressure-sensitive valve may include a linear slit within the distal end of the tubular instrument. In some embodiments, the linear slit may extend through an annular wall of the tubular member. In some embodiments, the linear slit may be oriented parallel to a longitudinal axis of the tubular instrument. In some embodiments, at least a portion of the distal end of the tubular instrument may be constructed of silicon, polypropylene, or another suitable material. In some embodiments, the portion of the distal end may include the linear slit.

In some embodiments, a method of blood collection may include inserting the catheter into vasculature of a patient. In some embodiments, the catheter assembly may include the catheter adapter, and the catheter extending distally from the catheter adapter. In some embodiments, the method may include coupling the delivery device to the catheter adapter. In some embodiments, the delivery device may include the housing and the tubular instrument.

In some embodiments, the method may include advancing the tubular instrument through the catheter, which may include advancing the tubular instrument distally with respect to the housing. In some embodiments, after advancing the delivery device through the catheter, the method may include activating a blood collection container. In some embodiments, activating the blood collection container may include coupling an evacuated blood collection container to the delivery device. In some embodiments, activating the blood collection container may include coupling a syringe to the delivery device and pulling the syringe. In some embodiments, in response to activating the blood collection container, the predetermined pressure differential within the lumen may be created and the pressure-sensitive valve may move from the closed position to the open position.

In some embodiments, the tubular instrument may be resistant to occlusion and thrombosis because the pressure-sensitive valve may be closed and blood may not be allowed to diffuse into the tubular instrument under normal physiological pressures. Thus, in some embodiments, the catheter assembly through which the tubular instrument extends may be flushed less frequently, such as, for example, once per week, instead of, for example, once per shift of a clinician.

It is to be understood that both the foregoing general description and the following detailed description are exemplary and explanatory and are not restrictive, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example catheter system, illustrating an example tubular instrument in a proximal position, according to some embodiments;

FIG. 1B is an upper perspective view of the catheter system, illustrating the tubular instrument in a distal position, according to some embodiments;

FIG. 2A is an upper perspective view of an example delivery device of the catheter system, illustrating the tubular instrument in the proximal position, according to some embodiments;

FIG. 2B is an upper perspective view of the delivery device of the catheter system, illustrating the tubular instrument in the distal position, according to some embodiments;

FIG. 3A is an upper perspective view of an example distal portion of the tubular instrument, illustrating an example slit in a closed position, according to some embodiments;

FIG. 3B is an upper perspective view of the distal portion of FIG. 3A, illustrating the slit in an open position during infusion, according to some embodiments;

FIG. 3C is an upper perspective view of the distal portion of FIG. 3A, illustrating the slit in an open position during blood withdrawal, according to some embodiments;

FIG. 4A is a side view of another example distal portion of FIG. 3A, illustrating an example pressure-sensitive valve in a closed position, according to some embodiments;

FIG. 4B is a cross-sectional view of the distal portion of FIG. 4A, according to some embodiments;

FIG. 4C is a cross-sectional view of the distal portion of FIG. 4A, illustrating the pressure-sensitive valve in an open position, according to some embodiments;

FIG. 4D is an upper perspective view of the distal portion of FIG. 4A, according to some embodiments; and

FIG. 4E is an upper perspective view of another example distal portion, according to some embodiments.

### DESCRIPTION OF EMBODIMENTS

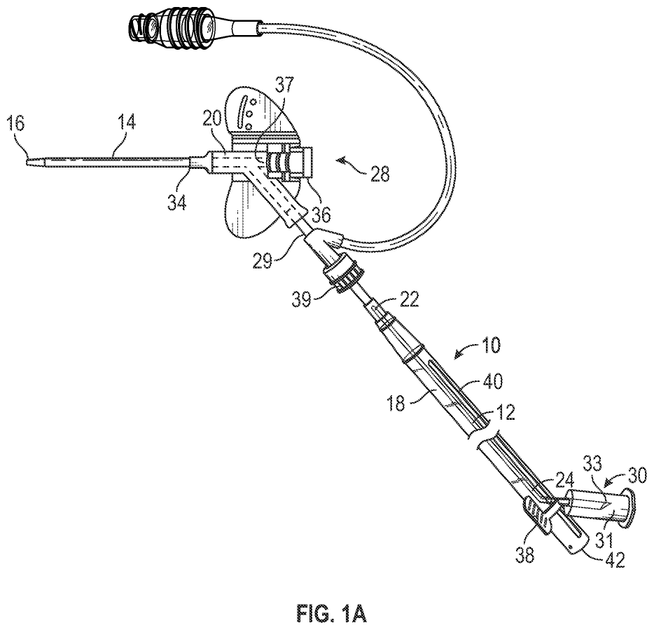

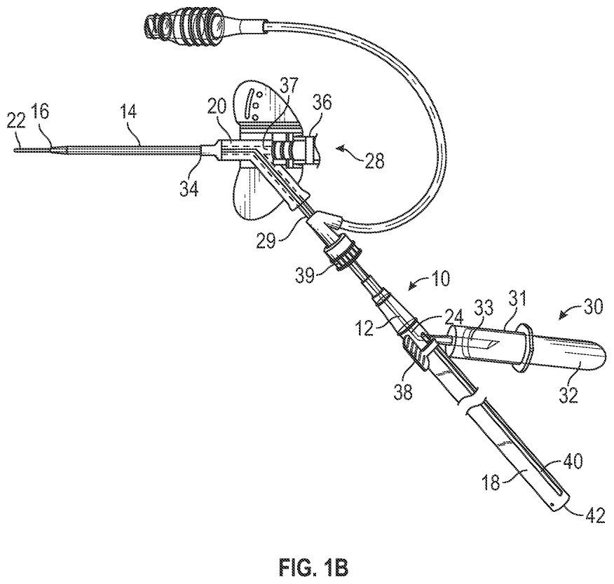

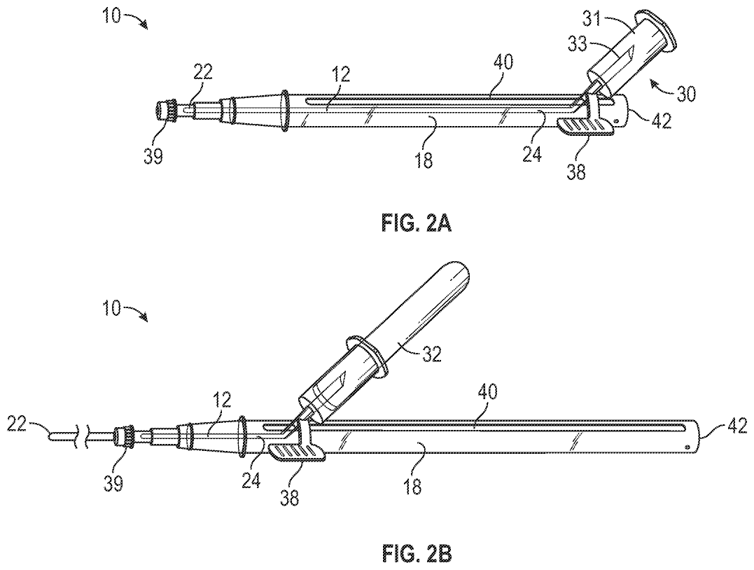

Referring now to FIGS. 1A-2B, in some embodiments, a delivery device 10 to deliver a tubular instrument 12 into a catheter 14 may facilitate an increased dwell period of the catheter 14. In further detail, the delivery device 10 may be used to advance the tubular instrument 12 into the catheter 14 and/or beyond a distal tip 16 of the catheter 14 for fluid infusion or blood draw when the catheter 14 is compromised or nearing an end of its life. In some embodiments, the tubular instrument 12 may be configured to reduce trauma to a vein of a patient upon contact with the vein of the patient, compared to prior art devices. In some embodiments, the tubular instrument 12 may provide gentle, soft contact between the tubular instrument 12 and a vein wall, which may reduce trauma to the vein wall. In some embodiments, the tubular instrument 12 may include another catheter. In some embodiments, the tubular instrument 12 may include a probe.

In some embodiments, the delivery device 10 may include a housing 18 configured to couple to a catheter adapter 20. In some embodiments, the delivery device 10 may include the tubular instrument 12, which may include a distal end 22, a proximal end 24, and a lumen extending between the distal end 22 and the proximal end 24. In some embodiments, the proximal end 24 of the tubular instrument 12 may be secured within the housing 18. In some embodiments, the tubular instrument 12 may be disposed within the housing 18 and/or configured to advance distally with respect to the housing 18.

In some embodiments, the delivery device 10 may include any suitable delivery device. Non-limiting examples of delivery devices that may be used with the tubular instrument 12 are described further in in U.S. patent application Ser. No. 16/037,246, filed Jul. 17, 2018, entitled “EXTENSION HOUSING A PROBE OR INTRAVENOUS CATHETER,” U.S. patent application Ser. No. 16/388,650, filed Apr. 18, 2019, entitled “INSTRUMENT DELIVERY DEVICE HAVING A ROTARY ELEMENT,” U.S. patent application Ser. No. 16/037,319, filed Jul. 17, 2018, entitled “MULTI-DIAMETER CATHETER AND RELATED DEVICES AND METHODS,” U.S. patent application Ser. No. 16/502,541, filed Jul. 3, 2019, entitled “DELIVERY DEVICE FOR A VASCULAR ACCESS INSTRUMENT,” U.S. patent application Ser. No. 16/691,217, filed Nov. 21, 2019, entitled “SYRINGE-BASED DELIVERY DEVICE FOR A VASCULAR ACCESS INSTRUMENT,” U.S. patent application Ser. No. 16/742,013, filed Jan. 14, 2020, entitled “CATHETER DELIVERY DEVICE AND RELATED SYSTEMS AND METHODS,” and U.S. patent application Ser. No. 16/838,831, filed Apr. 2, 2020, entitled “VASCULAR ACCESS INSTRUMENT HAVING A FLUID PERMEABLE STRUCTURE, AND RELATED DEVICES AND METHODS,” which are each incorporated by reference in their entirety.

In some embodiments, in response to the tubular instrument 12 being advanced distally with respect to the housing 18, the delivery device 10 may be configured to introduce the tubular instrument 12 into a catheter assembly 28, which may include the catheter adapter 20 and the catheter 14. In some embodiments, when the tubular instrument 12 is introduced into the catheter assembly 28, the tubular instrument 12 may access a fluid pathway of the catheter assembly 28 and/or the tubular instrument 12 may extend through the catheter assembly 28 to access vasculature of a patient.

In some embodiments, the catheter assembly 28 may include or correspond to any suitable catheter assembly, such as, for example, the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable catheter assembly. In some embodiments, the catheter assembly 28 may be integrated with an integrated extension tube 29. In other embodiments, the catheter assembly 28 may be non-integrated. In some embodiments, the catheter 14 may include a peripheral intravenous catheter (PIVC), a peripherally inserted central catheter (PICC), or a midline catheter.

In some embodiments, the catheter 14 may be secured within and extend distally from the catheter adapter 20. In some embodiments, the catheter adapter 20 may include a distal end 34, a proximal end 36, and a lumen 37 extending through the distal end 34 and the proximal end 36. In some embodiments, a septum may be disposed within the lumen of the catheter adapter 20. In some embodiments, the tubular instrument 12 may be delivered to the vasculature through the septum or proximal to the septum.

In some embodiments, the delivery device 10 may include an adapter 39, which may be coupled to the proximal end 36 or another portion of the catheter assembly 28, such as, for example, a Y-adapter. In some embodiments, the adapter 39 may include a slip or thread or clip male luer adapter. In some embodiments, the adapter 39 may include a slip or thread or clip female luer adapter.

In some embodiments, the delivery device 10 may include a blood collection device 30. In some embodiments, the blood collection device 30 may include or correspond to a blood collection container. In some embodiments, the blood collection container may include a syringe, an evacuated blood collection tube 32, a small sample collection device, or any other container configured to collect blood from a patient via a pressure differential.

In some embodiments, the blood collection device may include a needle assembly, which may include a needle 33 configured to receive the blood collection container. In some embodiments, a proximal tip of the needle 33 may be disposed within an elastomeric sheath. In some embodiments, in response to the blood collection container pushing the elastomeric sheath distally, the needle 33 may pierce the elastomeric sheath and be inserted into the blood collection container. In these and other embodiments, the blood collection container may include the evacuated blood collection tube 32.

In some embodiments, the blood collection device may include a holder 31, which may be configured to receive the evacuated blood collection tube 32. In some embodiments, the blood collection device may include the Becton Dickinson VACUTAIINER® one-use holder. In some embodiments, the blood collection device 30 may be coupled to and in fluid communication with the proximal end 24 of the tubular instrument 12. In some embodiments, the blood collection device 30 may be coupled to and in fluid communication with the proximal end 24 of the tubular instrument 12 via a fluid pathway extending through the needle 33 and the tubular instrument 12. In some embodiments, the blood collection device 30 may be coupled to the proximal end 24 of the tubular instrument 12 in any number of suitable ways, such as via integration, a Luer connection, etc.

In some embodiments, the delivery device 10 may include an advancement element, such as a tab 38 or a grip, which may be moved by the clinician to advance the tubular instrument 12 in a distal direction and/or retract the tubular instrument 12 in a proximal direction. In some embodiments, the advancement element may be coupled to the tubular instrument 12. In some embodiments, the advancement element may be rotated. In some embodiments, the advancement element may be moved along a slot 40 in the housing 18, as illustrated in FIGS. 1A-1B. In some embodiments, the tubular instrument 12 may extend and move through a proximal end 42 of the housing 18.

In some embodiments, in response to significant dwelling time within the vasculature, the catheter 14 of the catheter assembly 28 may be susceptible to narrowing, collapse, kinking, blockage by debris (e.g., fibrin or platelet clots), and adhering of the distal tip 16 of the catheter 14 to the vasculature. Thus, blood withdrawal using the catheter 14 may be difficult. In some embodiments, the tubular instrument 12 may include or act as another catheter that may provide access to the vasculature of the patient without any additional needle sticks without any additional needle sticks. Thus, in some embodiments, the tubular instrument 12 may be used for needle-free blood collection and/or fluid infusion. In some embodiments, the tubular instrument 12 may include a pressure-sensitive valve, which may decrease a susceptibility of the tubular instrument 12 to occlusion and thrombosis during blood collection and/or fluid infusion.

In some embodiments, a method of blood collection may include inserting the catheter 14 into vasculature of a patient. In some embodiments, the method may include coupling the delivery device 10 to the catheter adapter 20. In some embodiments, the method may include advancing the tubular instrument 12 through the catheter 14, which may include advancing the tubular instrument 12 distally with respect to the housing 18. In some embodiments, after advancing the tubular instrument 12 through the catheter 14, the method may include activating the blood collection device 30.

In some embodiments, the tubular instrument 12 may include the probe. In these and other embodiments, the tubular instrument 12 may not be coupled to the blood collection device 30. In some embodiments, the probe may be configured to sense one or more conditions within the vein and/or the catheter assembly 28.

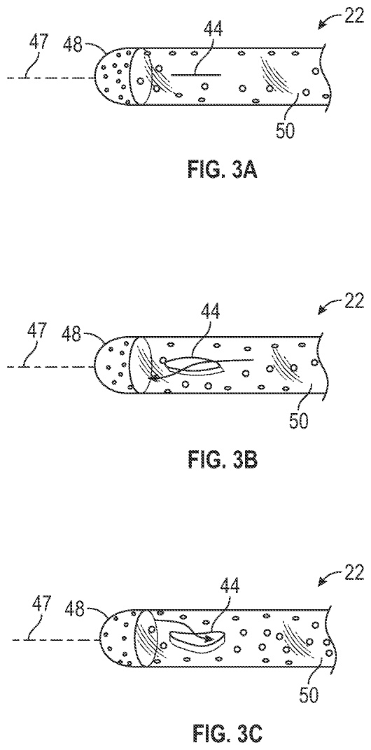

Referring now to FIGS. 3A-3C, in some embodiments, the pressure-sensitive valve 44 may be closed and blood may not be allowed to diffuse into the tubular instrument 12 under normal physiological pressures. Thus, in some embodiments, the catheter assembly 28 through which the tubular instrument 12 extends may be flushed less frequently, such as, for example, once per week, instead of, for example, once per shift of a clinician.

In some embodiments, the pressure-sensitive valve 44 may include a dynamic valve, which may open and/or close in response to a user input. For example, in response to activation of the blood collection device 30 (illustrated, for example, in FIGS. 1A-2B) by the user, the pressure-sensitive valve 44 may open. In some embodiments, activating the blood collection device 30 may include coupling an evacuated blood collection container to the delivery device 10. In some embodiments, activation of the blood collection device 30 may include coupling a syringe to the proximal end 24 of the tubular instrument 12 and pulling the syringe. In some embodiments, in response to activating the blood collection device 30, a predetermined pressure differential within the lumen of the tubular instrument 12 may be created and the pressure-sensitive valve 44 may move from a closed position to an open position.

In some embodiments, the pressure-sensitive valve 44 may include a linear slit. In some embodiments, the linear slit may be oriented parallel to a longitudinal axis 47 of the tubular instrument 12. In some embodiments, a distal-most portion of the distal end 22 may include a distal tip 48, which may be closed. In some embodiments, the distal tip 48 may be curved or rounded. In some embodiments, the distal tip 48 may close a distal end of the lumen of the tubular instrument 12. In some embodiments, the tubular instrument 12 may include an annular wall 50. In some embodiments, the linear slit may extend through the annular wall 50. In some embodiments, the annular wall 50 may be proximate the distal tip 48.

In some embodiments, the annular wall 50 may include a first material, and the distal tip 48 may include a second material. In some embodiments, the second material may have a lower durometer than the first material, which may facilitate softer contact between the tubular instrument 12 and a wall of the vasculature. In some embodiments, the tubular instrument 12 and/or a portion of the tubular instrument 12 that includes the linear slit may be constructed of silicon, polypropylene, or another suitable material.

In some embodiments, as illustrated, for example, in FIG. 3A, the linear slit may be in a closed position. In some embodiments, when the linear slit is in the closed position, opposing faces of the linear slit may contact each other. In some embodiments, the linear slit may be in the closed position and sealed under normal physiological pressures, preventing fluid from flowing through the linear slit. In some embodiments, the tubular instrument 12 may be resistant to occlusion and thrombosis because the linear slit may be closed under normal physiological pressures, preventing blood from diffusing into the tubular instrument 12. Thus, in some embodiments, the catheter assembly 28 (illustrated, for example, in FIGS. 1A-1B) that includes the tubular instrument 12 may be flushed less frequently, such as, for example, once per week, instead of, for example, once per shift of a clinician.

In some embodiments, in response to the predetermined pressure differential, the linear slit may open. In some embodiments, the linear slit may open during infusion of fluid into the patient, as illustrated, for example, in FIG. 3B. In some embodiments, the linear slit may open during withdrawal of blood from the patient, as illustrated, for example, in FIG. 3C.

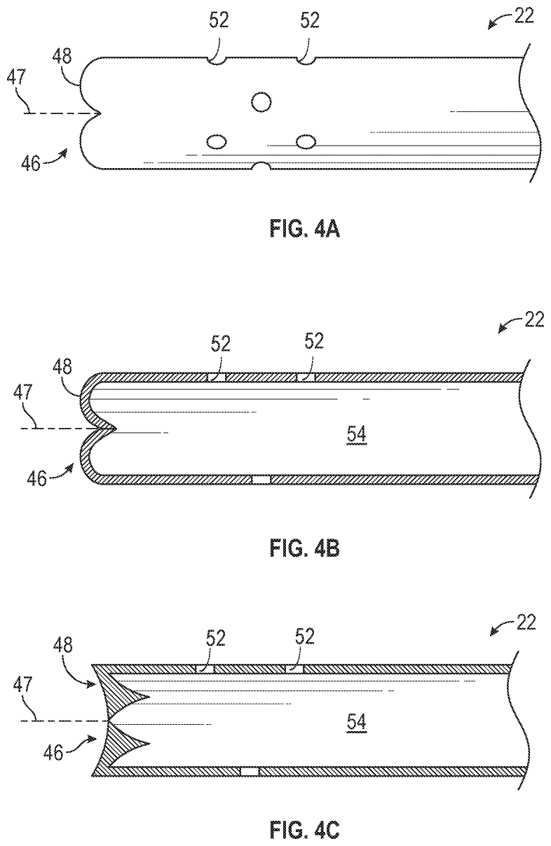

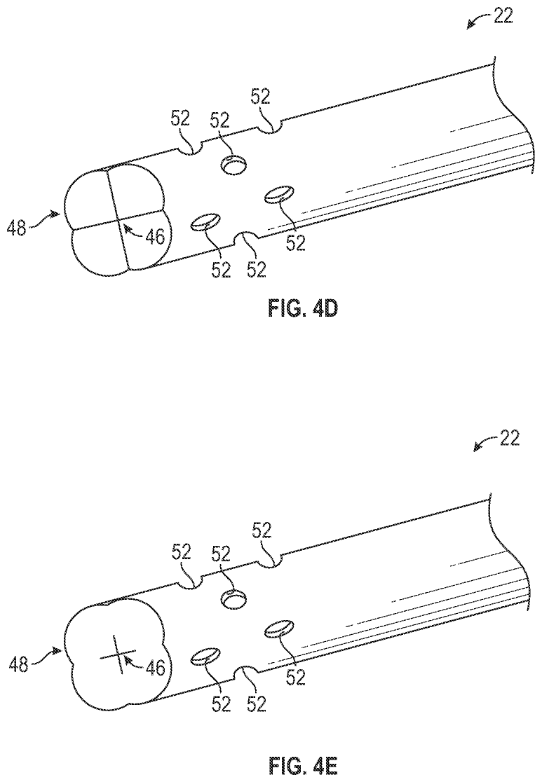

Referring now to FIGS. 4A-4E, a pressure-sensitive valve 46 may include a slit disposed within the distal end 22 of the tubular instrument 12. In some embodiments, the pressure-sensitive valve 46 may be similar or identical to the pressure-sensitive valve 44 in terms of one or more included features and/or operation. In some embodiments, the pressure-sensitive valve 46 may be configured to move from a closed position to an open position in response to a vacuum pressure within the lumen of the tubular instrument 12 being within a predetermined pressure window. In further detail, in some embodiments, the pressure-sensitive valve 46 may open in response to the vacuum pressure is above a predetermined level and below another predetermined level. In some embodiments, the vacuum pressure may be created in response to activating the blood collection device 30. In some embodiments, the pressure-sensitive valve 46 may be in the closed position in response to the vacuum pressure being too high, which may reduce a likelihood of collecting a blood sample with damaged blood cells.

In some embodiments, the pressure-sensitive valve 46 may be disposed at the distal tip 48. In these and other embodiments, the pressure-sensitive valve 46 may include a slit. In some embodiments, the pressure-sensitive valve 46 may include a cross slit. In some embodiments, the cross slit may be x-shaped or t-shaped. In some embodiments, a length of the cross slit may extend across an entirety of a width of the distal end 22. In some embodiments, a length of the cross slit may extend only partially across a width of the distal end 22. In some embodiments, the tubular instrument may include multiple holes 52 within the distal end 22 and proximal to the pressure-sensitive valve 46. In some embodiments, the pressure-sensitive valve 46 may be in the closed position to prevent fluid from flowing through the lumen. In some embodiments, the pressure-sensitive valve 46 may be in the closed position under normal physiological pressures.

In some embodiments, the pressure-sensitive valve 46 may be monolithically formed with the tubular instrument as a single unit. In some embodiments, the pressure-sensitive valve 46 may include a septum, which may be secured within the lumen 54 of the tubular instrument 12. In some embodiments, the pressure-sensitive valve 46 and/or the holes 52 may be configured to reduce trauma to the vein of the patient upon contact with the vein of the patient. In some embodiments, the pressure-sensitive valve 46 and/or the holes 52 may reduce a stiffness of the distal end 22 of the tubular instrument 12 to provide gentle, soft contact between the tubular instrument 12 and the vein wall, which may reduce trauma to the vein wall. In some embodiments, the tubular instrument 12 may include the other catheter. In some embodiments, the tubular instrument 12 may include the probe.

All examples and conditional language recited herein are intended for pedagogical objects to aid the reader in understanding the present disclosure and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

1. A delivery device to deliver a tubular instrument into a catheter extending distally from a catheter adapter, the delivery device comprising:
a housing configured to couple to the catheter adapter;
the tubular instrument configured to insert through the catheter, the tubular instrument comprising a distal end, a proximal end, and a lumen extending between the distal end and the proximal end, wherein the distal end comprises a distal tip, wherein the distal tip is closed, wherein the tubular instrument is configured to advance distally with respect to the housing; and
a pressure-sensitive valve disposed at the distal tip, wherein the pressure-sensitive valve is configured to move from a closed position to an open position in response to a pressure differential within the lumen being within a predetermined pressure window.

2. The delivery device of claim 1, wherein the tubular instrument comprises a plurality of holes within the distal end and the proximal to the pressure-sensitive valve.

3. The delivery device of claim 1, wherein the pressure-sensitive valve is monolithically formed with the tubular instrument as a single unit.

4. The delivery device of claim 1, wherein the pressure-sensitive valve comprises a slit.

5. The delivery device of claim 1, wherein the pressure-sensitive valve comprises a cross slit.

6. The delivery device of claim 5, wherein a length of the cross slit extends across an entirety of a width of the distal end.

7. The delivery device of claim 5, wherein a length of the cross slit extends only partially across a width of the distal end.

8. A delivery device to deliver a tubular instrument into a catheter extending distally from a catheter adapter, the delivery device comprising:
a housing configured to couple to a catheter adapter;
the tubular instrument configured to insert through the catheter, the tubular instrument comprising a distal end, a proximal end, a lumen extending between the distal end and the proximal end, and a linear slit within the distal end, wherein the distal end comprises a distal tip, wherein the distal tip is closed, wherein the tubular instrument is configured to advance distally with respect to the housing, wherein the linear slit is in a closed position, wherein the linear slit is configured to move from the closed position to an open position in response to a predetermined pressure differential within the lumen.

9. The delivery device of claim 8, wherein the linear slit is oriented parallel to a longitudinal axis of the tubular instrument.

10. The delivery device of claim 8, wherein the distal end of the tubular instrument is constructed of silicon.

11. The delivery device of claim 8, wherein the distal end of the tubular instrument is constructed of polypropylene.

12. A method of blood collection, comprising:
inserting a catheter of a catheter assembly into vasculature of a patient, wherein the catheter assembly comprises a catheter adapter and a catheter extending distally from the catheter adapter;
coupling a delivery device to the catheter adapter, wherein the delivery device comprises a housing and a tubular instrument, wherein the tubular instrument comprises a distal end, a proximal end, and a lumen extending through the distal end and the proximal end, wherein the distal end comprises a closed distal tip, wherein the proximal end is secured within the housing, wherein the tubular instrument is disposed within the housing and configured to advance distally with respect to the housing, wherein the delivery device comprises a pressure-sensitive valve disposed within the distal end and in a closed position, wherein the pressure-sensitive valve is configured to move from the closed position to an open position in response to a predetermined pressure differential within the lumen;
advancing the delivery device through the catheter, comprising advancing the tubular instrument distally with respect to the housing; and
after advancing the delivery device through the catheter, activating a blood collection container, wherein in response to activating the blood collection device, the predetermined vacuum pressure within the lumen is created and the pressure-sensitive valve moves from the closed position to the open position.

13. The method of claim 12, wherein the pressure-sensitive valve comprises a slit at the distal tip, wherein the slit is in the closed position to prevent fluid from flowing through the lumen.

14. The method of claim 12, wherein the tubular instrument comprises a plurality of holes within the distal end and the proximal to the slit.

15. The method of claim 12, wherein the slit comprises a cross slit.

16. The method of claim 12, wherein the pressure-sensitive valve comprises a linear slit oriented parallel to a longitudinal axis of the tubular instrument and within the distal end.

17. The method of claim 16, wherein the distal end of the tubular instrument is constructed of silicon.

18. The method of claim 16, wherein the distal end of the tubular instrument is constructed of polypropylene.
